(19) United States
(12) Plant Patent Application Publication
(10) Pub. No.: US 2014/0109278 P1
(43) Pub. Date: Apr. 17, 2014

(54) Lobelia plant named 'KLELE12472'

(50) ;

(71) Applicant: KLEMM+SOHN GMBH & CO. KG, Stuttgart (DE)

(72) Inventors: Nils Klemm, Stuttgart (DE); Guido Von Tubeuf, Stuttgart (DE)

(73) Assignee: KLEMM+SOHN GMBH & CO. KG, Sttuttgart (DE)

(21) Appl. No.: 13/573,956

(22) Filed: Oct. 16, 2012

(51) Int. Cl. A01H 5/00

(52) U.S. Cl. PLT/451

(57) ABSTRACT

A new and distict lobelia plant named ‘KLELE12472’ particularly distinguished by dark blue flowers and a medium compact, mounding growth habit, is disclosed.

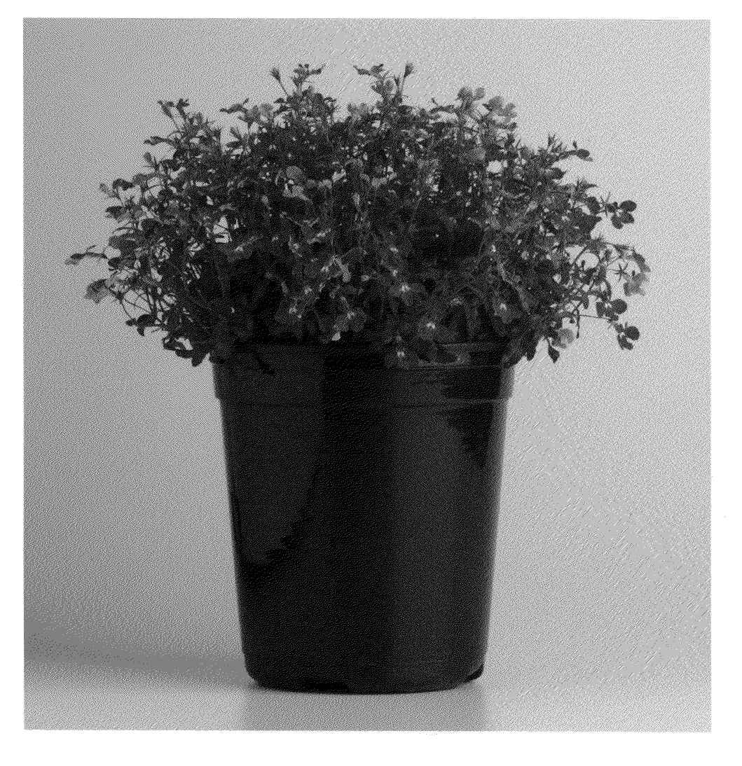

### GENUS AND SPECIES

Lobelia erinus

### VARIETY DENOMINATION

‘KLELE12472’

### BACKGROUND OF THE NEW PLANT

The present invention comprises a new and distinct variety of lobelia, botanically known as Lobelia erinus, and hereinafter referred to by the variety name ‘KLELE12472’. The new variety resulted from a controlled cross conducted in June 2006 in Stuttgart, Germany, between female parent lobelia plant named ‘LE 06 0012’ (unpatented) and male parent lobelia plant named ‘LE 06 0028’ (unpatented). A single plant selection was subsequently chosen for further evaluation and asexual propagation.

The new variety was first propagated via vegetative cuttings and in vitro propagation in May 2007 in Stuttgart, Germany, and has been asexually reproduced repeatedly by vegetative cuttings for 15 generations. ‘KLELE 12472’ has been found to retain its distinctive characteristics through successive asexual propagations via vegetative cuttings.

Plant Breeder's Rights for this variety were applied for in Canada on Feb. 17, 2012. ‘KLELE 12472’ has not been made publicly available anywhere in the world more than one year prior to filing of this application.

### SUMMARY OF THE INVENTION

The following are the most outstanding and distinguishing characteristics of this new variety when grown under normal horticultural practices in a glass greenhouse in Stuttgart, Germany.

1. Dark blue flowers; and

2. Medium compact, mounding growth habit.

### DESCRIPTION OF PHOTOGRAPH

This new lobelia plant is illustrated by the accompanying photograph which shows overall plant habit including blooms, buds, and foliage of the plant in full color. The colors shown are as true as can be reasonably obtained by conventional photographic procedures. The photograph was taken in spring of 2012 on a 3-month old lobelia plant grown in a 10 centimeter pot in a glass greenhouse in Stuttgart, Germany under normal horticultural practices.

### DESCRIPTION OF THE NEW VARIETY

The following detailed descriptions set forth the distinctive characteristics of ‘KLELE12472’. The data which define these characteristics were collected from asexual reproductions carried out in Stuttgart, Germany. The plant history was taken in the spring of 2012 on 3-month old plants grown in 10 centimeter pots in a glass greenhouse with a pinch date at week eight. Color readings were taken under natural light. Color references are primarily to the R.H.S. Colour Chart of The Royal Horticultural Society of London (R.H.S.), Fifth Edition (2007).

### DETAILED BOTANICAL DESCRIPTION OF THE NEW PLANT

Classification:
   Botanical.—Lobelia erinus.
   Common name.—Lobelia.
   Designation.—‘KLELE12472’
Parentage:
   Female parent.—Lobelia plant named ‘LE 06 0012’ (unpatented).
   Male parent.—Lobelia plant named ‘LE 06 0028’ (unpatented).
Growth:
   Growth and branching habit.—Mounding.
   Form.—Rounded to flat.
   Height (measured from the top of the soil, including any flowers).—15.0 cm.
   Width (horizontal plant diameter, including any flowers).—12.0 cm.
   Time to produce a finished flowering plant.—9 weeks.
   Outdoor plant performance.—Very good.
   Time to initiate roots.—3 weeks.
   Root description.—Fine, white roots, freely branching and dense.
Leaves:
   Arrangement.—Alternate.
   Length.—2.0 cm to 2.5 cm.
   Width.—0.3 cm to 0.4 cm.
   Shape.—Elliptical.
   Margin.—Serrate to lanceolate.
   Apex.—Obtuse.
   Base.—Acute.
   Texture (both upper and lower surfaces).—Smooth.
   Color (immature and mature leaves).—Upper surface: RHS 137A Lower surface: RHS 137C.
   Venation.—Pinnate.
Stems:
   Length.—12.0 cm.
   Diameter.—0.2 cm.
   Internode length.—1.0 cm to 1.5 cm.
   Color.—RHS 137B.
   Texture.—Smooth.
   Stem anthocyanin.—Absent.
   Peduncle.—Color: RHS 137B Length: 1.0 cm to 3.0 cm Diameter: 0.05 cm Texture: Smooth.
Flower buds:
   Color (when first opening).—Greyish violet.
   Shape.—Inverse drop.
   Diameter.—0.25 cm.
   Length.—0.8 cm.
Inflorescence:
   Blooming habit.—Continuous.
   Lastingness of individual blooms on plant.—Approximately 1 week.
   Fragrance.—Absent.
   Inflorescence type.—Raceme.
   Quantity of flowers and buds per flowering branch.—10 to 15.
Flowers:
   Arrangement.—Flowers form at apical axils, one flower per axil.
   Flower diameter (horizontal).—1.5 cm.
   Flower height (vertical).—0.6 cm.
   Immature flower.—Upper lobes color: Upper surface: Close to RHS N89A Lower surface: RHS 95C on the outer region and RHS 97D on the central region Lower lobes color: Upper surface: Close to RHS N89A Lower surface: RHS 94A.
   Mature flower.—Upper lobes: Color: Upper surface: Close to RHS N89A Lower surface: RHS 95C on the outer region and RHS 97D on the central region Length: 0.9 cm Width: 0.6 cm Shape: Obovate Apex: Obtuse Margin: Entire Texture (both upper and lower surfaces): Smooth Lower lobes: Color: Upper surface: Close to RHS N89A Lower surface: RHS 94A Length: 0.4 cm Width: 0.15 cm Shape: Obovate Apex: Obtuse Margin: Entire Texture (both upper and lower surfaces): Smooth.
   Corolla.—Color inside: RHS 94A; white with blue pattern Color outside: Close to RHS 94A; white with weak blue pattern Tube length: 0.7 cm Diameter (at top of corolla tube): 0.2 cm.
   Calyx.—Quantity of sepals: 5 Color: Upper surface: RHS 137A (fused part) and slight RHS 141C anthocyanin coloration near margins Lower surface: RHS 137A with slight anthocyanin coloration near margins Length: 0.9 cm Fused part: 0.4 cm Non-fused part: 0.5 cm Diameter (at fusion point): 0.1 cm Shape: Funnel, star-shaped with fused sepals in lower half Apex: Acute Texture (both upper and lower surfaces): Smooth.
Reproductive organs:
   Androecium.—Quantity of anthers: 5 Filament length: 0.7 cm Filament color: Bluish white Pollen color: Grey Pollen amount: Medium.
   Gynoecium.—Pistil length: 0.5 cm Stigma color: Greyed violet Style length: 0.6 cm Style color: RHS 142B.
Fruit and seed set: None observed.
Disease and insect resistance: Typical of species

### COMPARISON WITH PARENTAL AND COMMERCIAL VARIETIES

‘KLELE12472’ differs from the female parent, ‘LE 06 0012’, (unpatented) in that ‘KLELE12472’ has dark blue flowers, whereas ‘LE 06 0012’ has medium blue flowers with a white eye.

‘KLELE12472’ differs from the male parent, ‘LE 06 0028’, (unpatented) in that ‘KLELE12472’ has a mounding, medium compact habit, whereas ‘LE 06 0028’ has an upright, compact habit.

‘KLELE12472’ differs from the commercial variety ‘Tech Hepdab’ (U.S. Plant Pat. No. 21,049) in that ‘KLELE12472’ has a mounding, medium compact growth habit, whereas ‘Tech Hepdab’ has a half-hanging growth habit and is more vigorous.

We claim:

1. A new and distinct lobelia plant named ‘KLELE12472’ as shown and described herein.
